(19) United States
(12) Patent Application Publication
(10) Pub. No.: US 2012/0289942 A1
(43) Pub. Date: Nov. 15, 2012

(54) AUTOMATIC BARRIER FILM REMOVAL

(75) Inventors: Kim Becker, Hilleroed (DK); Jesper Mads Bartroff Frederiksen, Vedbaek (SE); Bent Ekfeldt, Koebenhavn V (DK); Kaspar Matthison-Hansen, Aalsgaarde (DK)

(73) Assignee: COLOPLAST A/S, Humlebaek (DK)

(21) Appl. No.: 12/993,514

(22) PCT Filed: May 20, 2009
(86) PCT No.: PCT/DK2009/050114

§ 371 Date: Apr. 5, 2011

(30) Foreign Application Priority Data
May 21, 2008 (DK) PA 2008 00697

(51) Int. Cl. A61M 27/00; B65B 55/08

(52) U.S. Cl. 604/544; 53/428

(57) ABSTRACT

An assembly (100) for wetting a medical device (101) is provided. The assembly has a package (110) for a medical device (such as a catheter) and a compartment (155) for a fluid medium (such as a saline solution containing an antimicrobial agent). This assembly includes a device (160) for removing covering parts of a barrier-film The covering parts (171,172) of the barrier film are used to cover the inlet (130) to the package and the outlet (149) from the compartment prior to use. The barrier-film (170) also includes a pulling part (373,573) which is used for pulling at the covering parts. The film-removal device uses different mechanisms to remove the film For example a toothing mechanism (163,164), a swivel arm (361), a wedge-mechanism (561) and a screw-and-spindle mechanism (761) can be used. All mechanisms are able to transfer a movement in one direction for connecting the inlet with the outlet into a pulling force in a transverse direction for removing the covering parts of the barrier film This way the user only needs to apply pressure in one direction to get the assembly ready for use. The mechanisms may all include a reel, which is used for winding the pulling part of the barrier film around it. Furthermore, a method for wetting a medical device and a method for manufacturing an assembly are provided.

This application concerns removal of barrier films in connection with an assembly for wetting a medical device, the assembly of which comprises a compartment for containing the fluid medium and a package for the medical device.

### BACKGROUND

Urinary catheters are widely used for intermittent catherisation, especially in connection with spinal cord injuries, where the user is left without control of the bladder. To reduce the risk of damage to the urethral wall, the catheters are typically coated with a coating, imparting an extremely low friction on the surface of the catheters. This coating is normally activated by applying a fluid medium (for example tap water or sterilised water) to the coating, either in the production stage or immediately prior to use. If the coating is activated prior to use, the fluid medium may be provided in a separate compartment with the medical device, thus forming an assembly comprising a package for the medical device and a compartment for the fluid medium. Such assemblies are shown in for example WO2006/092150. WO2006/092150 provides an assembly for preparing a medical device, in particular a urinary catheter, by releasing a fluid medium onto the device. The device is packed in a package, which contains the fluid medium confined in a compartment. DE10334372 provides a catheter system for urine withdrawal, comprising a tube for inserting into the human or animal body, which is provided with a coating that can be activated by means of liquid, and a compartment for containing the liquid, the compartment of which contains a disinfectant dissolved in the liquid.

Some users of catheters experience Urinary Tract Infections (UTI) very often. 30% experience more than three UTI's per year and 10% experience more than six UTI's per year.

To reduce risk of infection, the medical device as well as the fluid medium may be sterilised. This is particularly the case if the fluid medium is prepacked with the medical device in an assembly. It may be beneficiary to be able to sterilise the medical device and the fluid medium separately, and then attach the closed package containing the medical device and the closed compartment containing the fluid medium to each other, once sterilisation has taken place. To keep the content sterilised, the inlet to the package and the outlet from the compartment may both be covered with a protective liner or barrier film prior to use. These barrier films should be removed prior to connecting the inlet and the outlet to each other, because it is important to keep the inside parts completely uncontaminated. Hence contamination of the inlet and the outlet should be avoided, and they should not be in contact with the outer side of the barrier film. Therefore, an easy way to remove the barrier film and get the fluid into the package is needed for such assemblies.

### SUMMARY OF THE INVENTION

The invention concerns an assembly for wetting a medical device comprising a compartment for the fluid medium and a package for the medical device. The package has an inlet and the compartment has an outlet, both of which are covered by covering parts of a barrier film prior to use. When the assembly is to be used, the outlet and the inlet need to be brought into contact with each other. The invention provides film-removal devices, which are able to use the movement for bringing the outlet and the inlet together to simultaneously remove the covering parts of the barrier film from the inlet and the outlet. Then a user only needs to move the compartment and the package towards each other to connect the outlet and the inlet and during the same movement, remove the covering parts of the barrier film.

Furthermore, the invention provides methods of wetting a medical device using an assembly that includes a film-removal device. Finally, the invention provides methods of assembling an assembly that includes a film-removal device.

### DETAILED DESCRIPTION OF THE INVENTION

In a first aspect of the invention, the invention relates to an assembly for wetting a medical device comprising
a compartment for a fluid medium having an outlet,
a package for containing the medical device comprising an inlet for the fluid medium,
where the package and the compartment are separate elements which are joined together,
the outlet and inlet are prior to use covered by covering parts of a barrier film,
the assembly further comprises a film-removal device adapted to transfer a movement for bringing the outlet and inlet into contact with each other into a pulling force for removing the covering parts,
wherein the pulling force is exerted in a direction substantially transverse to the movement.

By having such a device it is possible, in one step, to remove the barrier film as well as connect the outlet and the inlet. Furthermore, the removal may be done by pressing on either side of the upper part of the assembly (either on the compartment or on the upper part of the package). For some users, for example users with poor hand dexterity, it is easy to apply pressure in one direction. They only need to support the assembly against a surface, and then they are able to remove the protective liners and get the fluid medium to enter the package by applying pressure to the assembly. Pressure is applied by using for example the underarm or the hand.

The fluid medium may be water or a saline solution, for example physiological 0.9% saline solution. In an embodiment, it may include an anti-microbial such as for example hydrogen peroxide. Using hydrogen peroxide provides an anti-microbial effect to the medical device, which helps prevent infections.

The assembly comprises a package and a compartment, which is two separate parts that are attached together. Prior to use, the fluid medium is stored in the compartment. The package comprises an inlet, and the compartment comprises an outlet for the fluid medium. The outlet and the inlet may be moved from a position from where they are out of contact with each other to a position where the outlet and inlet are in contact with each other. In the first position, the assembly is in a storing-position, and in the second connected position, the assembly is in a use-position. In a storing-position the assembly is configured to be stored, and in a use-position the assembly is ready to be used. The movement from a storing-position to a use-position may be done by moving the compartment and the package around a hinge element which connects the two parts together. The hinge may be in the form of a regular hinge pin and hinge flanges. This provides for a strong and durable hinge joint. The hinge may also be in the form of a foil hinge, or it may be made of material that is thinner than the surrounding material. Finally, the hinge may be provided by a click-connection where one part comprises a groove and the other the hinge pin. The two parts are snapped together and following that, they are able to pivot with respect to each other.

Typically, the top of the assembly is defined as the end comprising the hinge, and the bottom as the bottom of the package. The bottom of the compartment corresponds to the end opposite the hinged end.

The assembly of this invention may be used to store a urinary catheter with a hydrophilic coating. A hydrophilic-coated catheter needs to be wetted prior to use to activate the coating (by swelling). Hence storing the catheter in an assembly including the fluid medium as well, removes the need for a user to have access to clean water when the catheter is to be used.

The compartment for the fluid medium and the package for the medical device are separate elements, and therefore they may be particularly useful when the medical device is a urinary hydrophilic coated catheter, and when the fluid medium includes an antimicrobial agent in form of hydrogen peroxide. In that case, the sterilisation of the catheter and the fluid medium may be done separately, and then the sterile products may be joined at a later time.

It may be an advantage to extrude the lower part of the package. Then, it just needs a closing at the lower end to make the final lower part of the package. Materials such as PVC (Poly-Vinyl-Chloride), PU (Poly-Urethane), silicone or a styrene-based material are suitable for this use. Alternatively, the package may be made of foil, which is welded along the sides. For this purpose, materials such as aluminium, PETP (Poly-Ethylene-Tere-Phtalate), LDPE (Low-Density Poly-Ethylene), HDPE (High-Density Poly-Ethylene), PP (Poly-Propylene), PVC, PA (Poly-Amide), PET (Amorphous Polyester), and surface treated paper are suitable.

The upper part of the package needs to be rather rigid to include the film-removal device. This part may be made by injection moulding. Materials suitable for this comprise polyolefins in general, particularly PP, PE (Poly-Ethylene) and materials such as ABS (Acrylonitrile-Butadiene-Styrene), PC (Poly-Carbonate), silicone, and styrene-based materials.

By making the package of two parts, an upper rigid part and a lower foil part, the part of the package including the inlet is protected from being broken if the assembly were subjected to a pressure, for example during transport. However, for packing purposes, it may be an advantage that a part of the package is flexible, as this allows the assemblies to be packed closer together. In an embodiment, the lower part of the package is also made by injection-moulding using a rather rigid material. This has production advantages, because the entire package may be made in one step.

The compartment may also be made of an injection-moulded element. In this case, useful materials are polyolefins in general, particularly PP, PE, for example HDPE, and materials such as ABS, PC, silicone, and styrene-based materials. An injection-moulded element provides for a relatively rigid element, which is an advantage for use in connection with the film-removal device.

The fluid medium may be stored directly in the compartment. In that case, the compartment may comprise sealing material at the edges to provide for a liquid-tight enclosure.

For some embodiments, the compartment comprises a pouch containing the fluid medium. This further secures against contamination or leakage of the fluid medium prior to use. The compartment may be made in one box-like element but in an embodiment it may also be made as an element that can be opened. For example, the bottom part of the compartment may be entirely detachable by a snap-fit connection. Alternatively, two parts of the compartment may be openable along one edge. Again, it may be an advantage to use a type of snap-fit device along this edge to open and close the compartment. A compartment that can be opened makes it possible to enter a pouch containing the fluid medium into the compartment. In this way, the compartment may be injection-moulded in one location and in another location the compartment may be opened and the pouch may be entered into and attached inside the compartment. Finally, the compartment may be closed and the pouch secured inside the compartment. The pouch may be attached inside the compartment with usual mechanical (snap-fit, push-fit couplings, and so on) or adhesive connections. Such connections are well known by a skilled person and will not be discussed further. The pouch may be attached to the compartment in the area near the outlet. Therefore, when the compartment is connected with the package, the pouch opening is ensured. In a related embodiment, the pouch is also connected to the compartment at the top of the pouch to prevent the pouch from collapsing inside the compartment. If the pouch were to collapse inside the compartment, some of the fluid medium may be prevented from exiting the pouch and entering into the package, ultimately leading to lack of liquid for wetting the catheter.

The outlet from the compartment inlet may comprise a stud while the package inlet comprises a membrane, or vice versa. In other words, the connection between the outlet and the inlet is in the form of a spike-element and a rupturable membrane, for example a weak point. The weak point at the inlet may be provided as a thinner part of material or it may be provided as another piece of material, which is softer than the remaining part of the package. By using such a spike element, it is very easy to make the connection between the outlet and the inlet. This leaves out the need for a complete match between the outlet and the inlet, because the spike element will have a certain margin into which it may be able to penetrate.

The outlet from the compartment may also comprise a cut-off double lumen tube having a fluid channel and an air channel, and the inlet to the package is a weak point. The cut-off double lumen tube will function as a spike element. A user need only press the two parts (the compartment and the package) together at the area near the outlet and the inlet. Then the fluid will flow from the compartment to the interior of the package. The inlet to the package may also comprise the cut-off double lumen tube (or stud) and then the compartment comprises the membrane. The membrane at the compartment may be distended to enhance the weakness of the membrane and to ensure that the entire membrane is removed upon penetration.

The film-removal device is adapted to remove the covering parts of a barrier film, initially covering the inlet to the package and the outlet from the compartment.

The barrier film comprises one or more pulling parts and covering parts for covering the inlet and the outlet respectively. The barrier film is made of a material, which is substantially unstretchable. The material should be balanced so that the pulling part of the film should be able to transfer a pulling force to the covering parts without stretching too much. An example of a material is polyester film of a thickness of 23·m, which may be silicone-treated. The thickness of the film may be between 10·m and 50·m, as this would provide for the necessary flexibility of the film. In an embodiment, the covering parts of the barrier film are made of a material, which is more rigid than the pulling part, for example the same film (polyester film) but of a thicker material, for example 200 to 300·m. The covering parts may also have a larger extent (for example 12 mm) in the transverse direction of the film (corresponding to the width direction of the package and the compartment) than the pulling parts so that the pulling part is narrower (for example 5 to 10 mm) than the covering parts.

When the assembly is to be used the package inlet needs to be moved into contact with the outlet from the compartment (that is moving the assembly from a storing-position to a use-position). The film-removal device includes a transfer-mechanism that is adapted to utilize or transfer this movement to simultaneously remove the covering parts of the barrier film. The removal of the covering parts is done by pulling at the pulling part of the barrier film, where the pulling is exerted by the film-removal device as the assembly is moved from a storing-position to a use-position.

The pulling force is exerted in a direction substantially transverse to the movement of the assembly from a storing-position to a use-position. In other words the, covering parts are removed in a direction substantially transverse to the movement of the inlet and the outlet.

The compartment and package may be hinged together. In such a case, the movement of the inlet and/or the outlet will form part of a circle arc. The hinge will constitute the centre for the circle arc. The pulling force may then be substantially in the radial direction. This may be either in a direction towards the hinge or away from the hinge. However the pulling force may also be in another direction substantially transverse to the circle arc.

Different film-removal devices are contemplated.

In one embodiment, the film-removal device comprises a toothing mechanism, which in a related embodiment comprises two sets of cooperating teeth. The sets of teeth are placed facing each other so that a tooth in one set corresponds to a gap between two teeth in the other set. The device functions so that when an element (for example a film) is placed between the teeth and a user pushes the teeth together, a pulling force will be transferred to the film in a direction substantially perpendicular to the direction of the teeth. In such a film removal device, a part of the film (the pulling part) will be entered in between the teeth, and further parts (the covering parts) will cover the inlet and outlet respectively. As the barrier film is substantially unstretchable the pulling part of the film will be able to exert a pull at the covering parts of the film. However, the pulling part of the film should not be too rigid as it then may be difficult to bend around the teeth. Furthermore, the friction between the pulling part of the film and the teeth should be rather low to enable entering of the film between the teeth. To reduce the friction, the pulling part of the film may be silicone-treated at the surface. In an embodiment, the pulling part is attached on the back side of the compartment.

The assembly may be configured so that one set of teeth is attached to the compartment and another set of teeth is attached to the package. The teeth are then attached at the rear of the package and the compartment. In an embodiment, two or more teeth have rounded tips facing each other. Thereby the risk of the film getting caught on the tips of the teeth is minimized, and the film may easily enter into the gap between the teeth. A further enhancement of this effect may be achieved by having two or more teeth comprising rolls at the tips facing each other. The pulling part of the film would then be able to roll into the gap over the tips or ends of the teeth.

The length of the teeth may be different in the two sets of teeth. In one set of teeth, the length of the individual teeth may be short enough to correspond to the wall thickness of the injection-moulded parts of the assembly. In other words, the device will function if one of the sets of teeth corresponds to the gaps for entering the other set of teeth. The length of the teeth may vary in at least one set of teeth. Initially there may be an angle between the compartment and the package. To initiate catching of the film as soon as the compartment and package are moved towards each other, the teeth near the bottom of the inlet may be longer than near the top. Then the pulling part of the barrier film is in contact with the teeth from both sides through the entire length of the pulling part. In an embodiment, the length of the teeth in one set of teeth is about twice as long near the bottom as it is near the top, and the length of teeth in the other set of teeth is substantially the same from top to bottom. Typically, the longest teeth are placed at the package, because there is less room for entering the teeth into the package due to the catheter that is placed here. The pouch may leave room for the teeth to enter into the compartment.

In another embodiment, the film-removal device comprises a swivel arm. As the compartment and the package are moved towards each other, the swivel arm will collapse or extend in a direction perpendicular to this movement. The pulling part of the film is attached to the swivel arm near the point, which in extended or collapsed position will be farthest from the inlet and the outlet. Thereby the movements of the swivel arm will be transferred to the pulling part of the film. The pulling part will then exert a pulling force at the covering parts of the film.

In an embodiment, the swivel arm comprises two rods hinged together, and in another embodiment, the swivel arm comprises more than two rods hinged together. The swivel arm needs at least two rods hinged together or a single rod, either bendable or hinged at the end, to be able to collapse or extend. More rods may be needed for some configurations of rods.

In an embodiment, the swivel arm extends between the compartment and the package so that a first rod is attached near the outlet from the compartment and a second rod is attached near the inlet to the package. The movement of the swivel arm may be upwards between the compartment and the package, downwards alongside the package, or outwards from the side of the package and the compartment. In another embodiment, a first rod is attached near the inlet to the package and a third rod is left unattached. This embodiment would require a shorter distance between the compartment and the package. Thereby the assembly in a storing-position would require less space.

Another embodiment of the invention relates to the film-removal device comprising a wedge mechanism. The wedge mechanism comprises a wedge and a sloped path for the wedge, having two sides at an oblique angle to each other. If the sides were parallel then the wedge may be jammed in between the sides, and make it impossible to move the wedge along the path. This is a simple and compact solution, which, prior to use, will give some structural stability to the assembly. The wedge may initially be carried by the barrier film. In an embodiment, the first and second side elements are integral with a back side of the compartment and a back side of the package. In another embodiment, the side elements may be made separately and then attached to the assembly at a later time. The pulling part of the barrier film is then attached to the wedge or will be pulled at, as the wedge is moved. As the compartment and the package are moved towards each other, the wedge mechanism will be moved due to the squeezing of the wedge-element. The pulling part of the film will exert a pulling force on the covering parts of the film, thereby eventually removing them from the inlet and the outlet.

In an embodiment, a cross-section of the wedge is shaped as an element having two opposite sides at an oblique angle to each other (for example a trapezoidal), or an element having tangential planes at two opposite sides at an oblique angle to each other (for example a cylinder or a spherical element). This is a very simple and compact solution. In another embodiment, a cross-section of the wedge is shaped as a triangle with wheels or rolls at the edges. Such a wedge may be very easy to move due the wheels or rolls. People with poor strength in their hands will be able to use such a mechanism. In a related embodiment, the wedge further comprises a reel. The pulling part of the film may then be rolled up around the reel and attached at the back side of either the compartment or the package. As the wedge is moved, the pulling part of the film is then moved twice the distance. Hence the distance the wedge should be removed before removing the covering parts of the film is halved.

The film-removal device may also comprise a screw-and-spindle-construction. A screw-and-spindle makes it possible to adjust the force needed to push the compartment and the package towards each other. If the angle of the screw thread (the lead angle) is rather low (for example 45°) compared to the length direction of the spindle, then the screw is easy to rotate along the spindle (it requires low pushing force) but it will need to travel a rather long distance along the spindle at each rotation. On the other hand, if the angle of the screw thread is close to 90° (for example 80°) with respect to the length direction of the spindle, then the screw is harder to rotate (it requires a higher pushing force) but it will only need to travel a short distance along the spindle at each rotation. To reduce the friction between the screw and the spindle, a ball-screw may be used.

In an embodiment, the spindle extends between the rear of the package and the rear of the compartment. This configuration correlates the movement of the compartment or package with the pushing force of the screw along the spindle. In a related embodiment, the screw rotates in planes substantially parallel to the backside of the compartment and the package. If the screw rotates in this direction, the configuration is quite compact, and therefore the assembly requires less space in the storing-position. The screw may also rotate in other directions, for example in planes that are substantially perpendicular to the backside of the compartment and the package. In this case, a rack and pinion device may be used.

All the above mentioned embodiments may benefit from the addition of a reel around which the pulling part of the barrier film is wound. This will double the length of film that is pulled away compared to the length of movement of the package and the compartment.

A resilient element may be placed surrounding the stud or spike at the inlet to the package. Such a resilient element will provide a contact face for the covering part of the film, which is to be attached at the stud or spike. As the spike is entered into the membrane, the resilient element will be compacted. The resilient element may be either a spring element or a sponge-like element.

Another aspect of this invention relates to a method for wetting a medical device stored in an assembly comprising a compartment for the fluid medium and a package for the medical device, where the compartment and the package are separate elements, which method comprises the steps of
moving the compartment and the package with respect to each other from a position where the assembly is in a storing-position to a position where the assembly is in a use-position, and
simultaneously removing covering parts of a barrier film from an inlet to the package and an outlet from the compartment, where the removing of the covering parts is in a direction transverse to the moving of the compartment.

Such a method requires only one movement by the user, and then the assembly is ready for use. It is only necessary to push two elements together, (the compartment and the package) and therefore this movement is easy for people with poor dexterity in their hands.

The covering parts may be removed with a toothing mechanism, a swivel arm, a wedge mechanism or a screw-and-spindle device as described earlier.

The method is particularly useful if the medical device is a catheter and the fluid medium includes an anti-microbial agent. Typically, the catheter needs to be wetted prior to use, and the fluid medium may be useful for this wetting. To reduce the risk of infections, it may be beneficial to use an anti-microbial agent in the fluid medium.

Yet another aspect of the invention relates to a method for manufacturing an assembly that comprises:
Assembling a package comprising an inlet and a part of a film-removal device and further including a medical device,
Producing a compartment comprising an outlet and a part of a film-removal device and further including the fluid medium,
Sterilising the compartment and the package separately from each other,
Joining the package and the compartment together,
Covering the outlet and the inlet with covering parts of a barrier film,
Attaching a pulling part of the barrier film to the film-removal device.

Such a method makes it possible to produce and sterilise the compartment and the package separately and then join the two parts together at another location. Thus it is possible to sterilise the package and the compartment at different degrees, which in some cases may be beneficial. Separating the compartment for a fluid medium and the package for containing the medical device is an advantage when considering sterilising the parts. Sterilisation of two smaller containers makes the dosage of the sterilisation medium easy to control, as the volume of each container is rather limited.

In an embodiment, the method concerns packing of a medical device, which is provided with a hydrophilic coating.

Separating the compartment and the package may also be an advantage due to the need to prevent medical devices with a hydrophilic coating from getting into contact with any fluid (for example moisture in the air) prior to packing. By separating the two containers, it is possible to produce and pack the medical device in one location and subsequently—without demands for packing in a controlled environment—shipping the package, including the medical device, to another location and here provide the fluid medium.

Ethylene-Oxide may be used to sterilise the package. Alternatively, it may be sterilised using radiation, for example beta- or gamma-radiation. By using these types of sterilisation, the package is ready to send, because it does not need time for evaporating after the sterilisation process.

The compartment may also be sterilised using radiation, for example beta- or gamma-radiation, or it may be sterilised using an autoclave treatment.

The joining may be done by attaching a hinge-part on the compartment to a hinge-part on the package. For example, it may be done by clicking a hinge pin on one part (the package or the compartment) into a groove at the other part (the package or the compartment). This is a simple and easy way of attaching the two parts together.

### BRIEF DESCRIPTION OF THE DRAWING

FIG. 1 illustrates an embodiment of the assembly including a film-removal device in the form of a toothing mechanism.

FIG. 2 illustrates an embodiment similar to the one in FIG. 1 except for the addition of a reel.

FIGS. 3 and 4 illustrate embodiments including a swivel arm.

FIGS. 5 and 6 illustrate embodiments including a wedge mechanism.

FIG. 7 illustrates an embodiment including a rack and pinion.

### DETAILED DESCRIPTION OF THE DRAWING

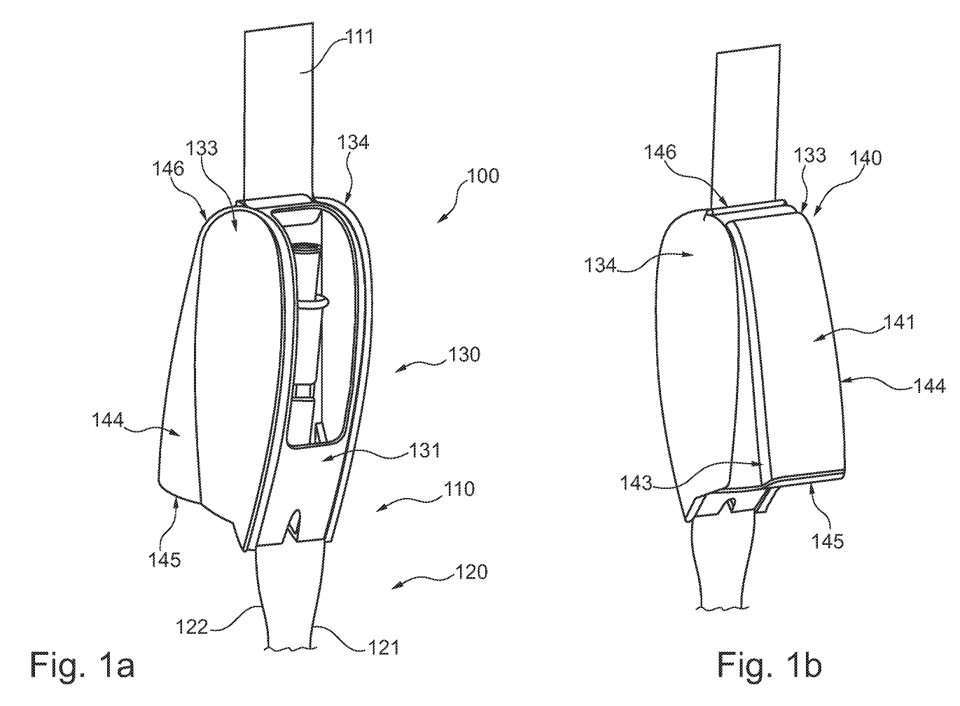

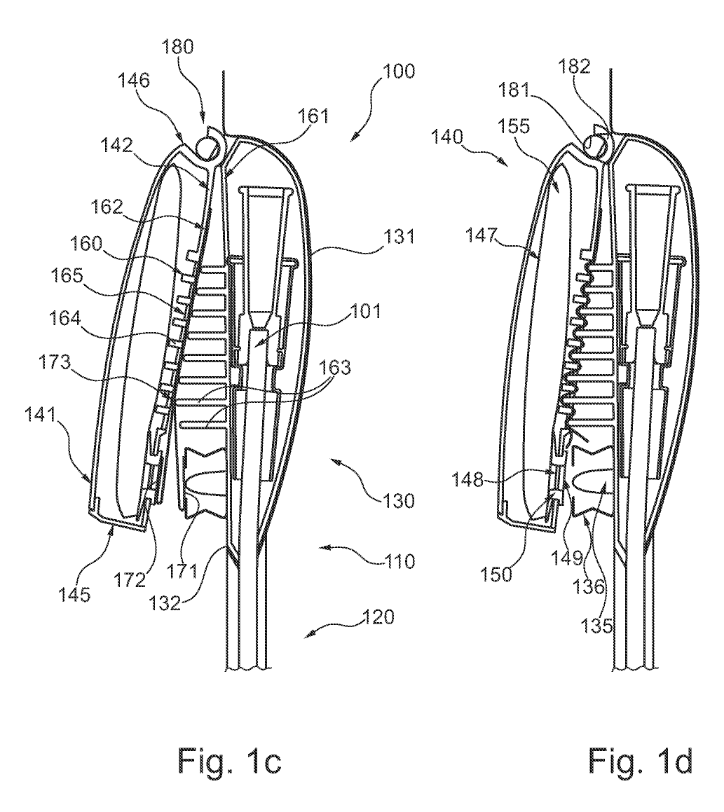

FIG. 1 illustrates an assembly 100 for wetting a medical device 101 (in this embodiment a catheter).The assembly 100 comprises a package 110 and a compartment 140 containing the fluid medium 155. FIGS. 1a and 1b illustrate 3D-views showing the front and the back of the assembly in a storing-position respectively. FIGS. 1c to 1e illustrate views with the side removed of the assembly in three different positions, in a storing-position, in a use-position and in an intermediate position. The package 110 consists of two parts: a lower foil part 120 and an upper more rigid part 130. In this embodiment, the lower part 120 of the package comprises two foils attached together along their sides as indicated at 121 and 122. Furthermore, the package is closed at the lower end (not shown). The upper part of the package 130 comprises a front side 131 facing away from the compartment 140 and a back side 132 facing the compartment, and two sides 133, 134 extending there between. The front side 131 comprises an opening strip 111 extending down along the front side and which may be removed to gain access to the medical device 101 contained in the package 110. The back side comprises a stud 135 for penetrating a membrane 148 on the compartment. The compartment has a front side 141 facing away from the package and a back side 142 facing towards the package, and two side-walls 143, 144 extending there between. In this embodiment, the compartment also has a bottom wall 145 and a top wall 146. The compartment 140 of this embodiment includes a pouch 147 in which the fluid medium 155 is located. In this embodiment, the pouch is attached to the compartment by adhesive connections at the top of the pouch and near the bottom of the pouch where the outlet 149 from the compartment is located. The membrane 148 is attached in an attachment device 150 in which the membrane is distended. The innermost surface of this attachment device may also be used as an attachment surface for the pouch in that it constitutes a plane surface for an adhesive connecting the pouch.

The compartment 140 and the upper part of the package 130 are joined together. In this embodiment this is done by a hinge element 180 comprising a hinge pin 181 and a groove 182 for receiving the pin 181. Clicking the hinge pin 181 into the groove 182 may assemble the assembly.

In this embodiment, the back side 132 of the upper part of the package and the back side 142 of the compartment each have a part of the film-removal device 160. The package part 161 and compartment part 162 each comprise teeth, which are adapted to interconnect upon moving the compartment to bring the assembly from a storing-position to a use-position. The teeth 163 of the package part 161 are of varying lengths so that the teeth are shorter towards the top of the assembly and longer towards the bottom of the upper part of the package. The teeth 164 on the compartment part 162 are more or less equally long over the entire compartment part 162. In the illustrated embodiment, the teeth extend inwards into the compartment, however they may also extend outwards, or they may be represented merely by gaps between elements corresponding to the material thickness of the compartment. Inwards-extending teeth apply strength to the relatively short teeth so that these are prevented from buckling during use due to the pressure from the film.

The barrier film 170 initially has a first covering part 171 covering the stud 135 at the package inlet 136, and a second covering part 172 covering the outlet 149 from the compartment. The two covering parts 171 and 172 are joined into a third pulling part 173 stretching up between the teeth. Because the compartment is moved to transfer the assembly from a storage position to a use position, the teeth 164 on the compartment will enter into the gaps between the teeth 163 on the package and thereby force the barrier film 170 in between the teeth. As the film is entered in between the teeth, the covering parts 171 and 172 are simultaneously removed from the inlet and outlet respectively by the pulling force exerted at the film. The film may be adhesively attached to the top part of the back side of the compartment, thereby ensuring that the film between the teeth is only pulled upwards. In this embodiment, the tooth at the top of the package is approximately 7 mm, the longest tooth near the bottom is approximately 12 mm, and the teeth on the compartment side are approximately 3 mm.

When the covering parts 171 and 172 of the barrier film 170 is entirely removed (as shown in FIG. 1d) the stud or spike 135 is ready to penetrate the membrane 148 and the fluid medium 155 may flow from the compartment 140 and into the package 110. FIG. 1e illustrates the assembly in the use-position. Finally, pulling the opening strip 111 may open the package, and the now wetted medical device 101 (catheter) may be removed and used.

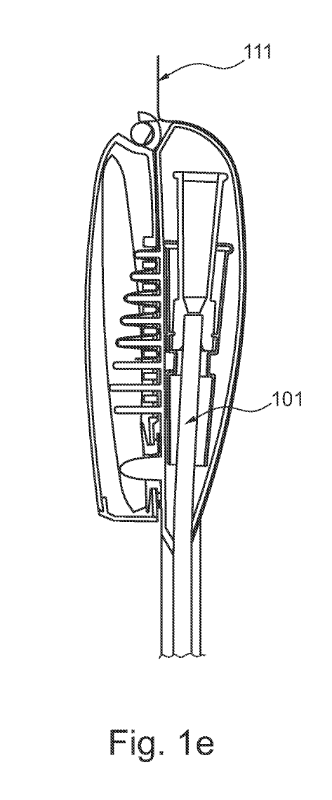

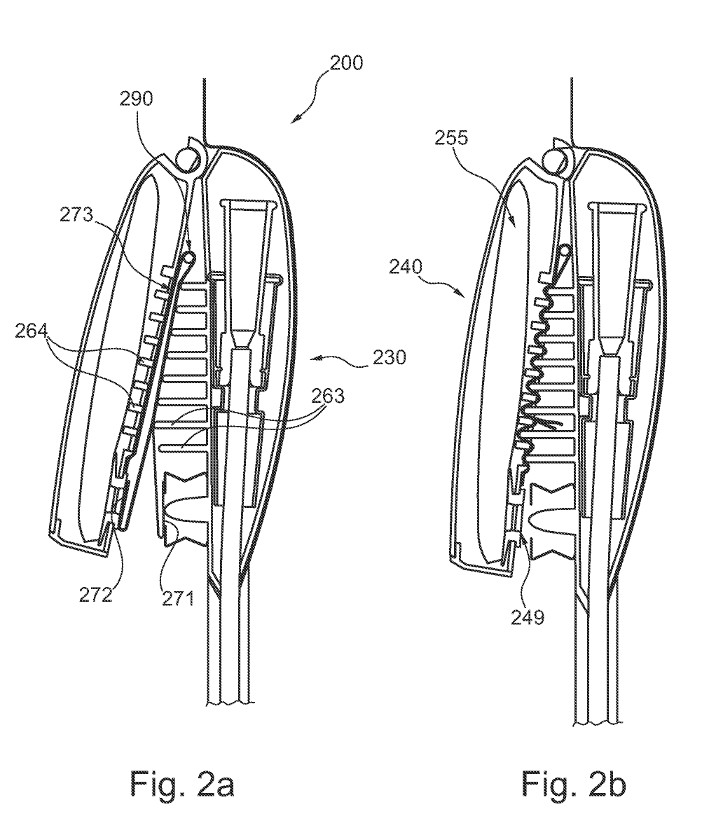

FIG. 2 illustrates an embodiment very similar to the one in FIG. 1. The same reference numbers are applied for the same elements except for the prefix 2. FIG. 2a illustrates the embodiment in the storing-position, and FIG. 2b illustrates the embodiment in an intermediate position where the covering parts 271, 272 of the barrier film are removed. The only difference is that in this embodiment the pulling part of the film 273 is approximately twice the length of the pulling part of the film of the embodiment in FIG. 1. This means that the pulling part of the film is long enough to stretch from the covering parts 271, 272 up between the teeth 263, 264 and around a reel 290 placed above the teeth. From there, the film stretches down between the teeth again, and finally the end of the pulling part is fastened to the compartment just above the outlet 249 from the compartment. This way of rolling the film has the advantage that the film will be influenced twice as much when it is forced in between the teeth. Thereby the distance the film has to move between the teeth could be halved.

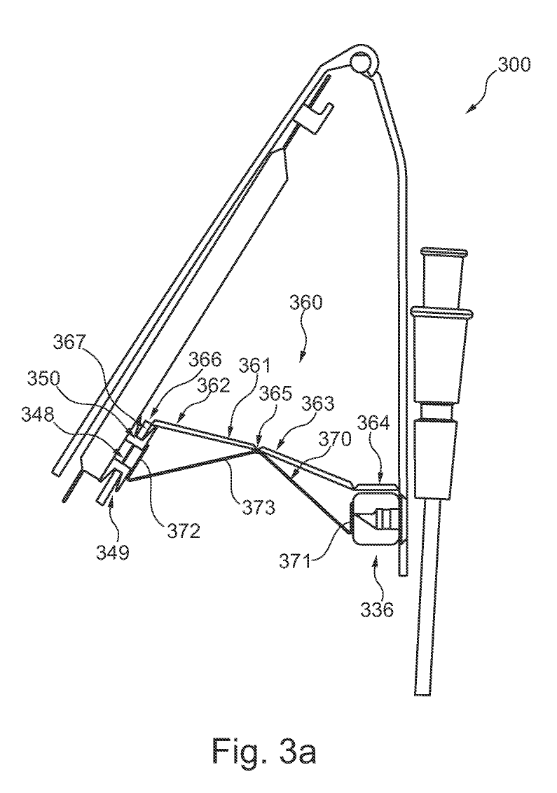

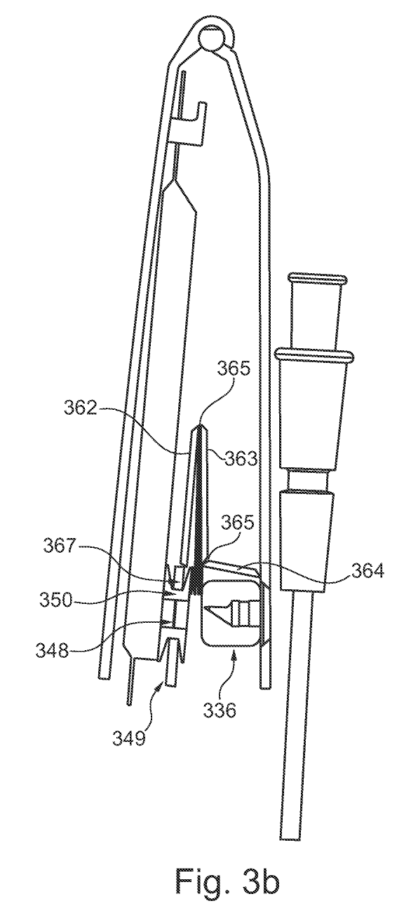

FIGS. 3a and 3b illustrate an embodiment with a different film removal device. Again, the same reference numbers are used for similar elements as in FIGS. 1 and 2 except for the prefix 3. Some parts of the assembly, which form no particular part of this embodiment, have been left out in FIGS. 3a and 3b. As an example, the front of the package of the assembly is not shown. The film removal device 360 of this embodiment comprises a swivel arm 361. Like with the earlier embodiments, the film 370 of this embodiment comprises two covering parts—a first covering part 371 for covering the inlet 336 to the package, and a second covering part 372 for covering the outlet 349 from the compartment. Furthermore, the film 370 comprises a pulling part 373 extending between the two covering parts. Initially, the compartment 340 and the package 330 are placed in a position leaving a distance between the inlet 336 to the package and the outlet 349 from compartment, which approximately corresponds to the length of the pulling part 373 of the film.

The swivel arm 361 comprises three rigid rods, which are hinged together. A first rod 362 extends from a hinging attachment on the compartment and approximately half the distance between the outlet and the inlet. A second rod 363 also extends approximately half the distance and is hingedly attached to the package. The two rods are hinged together by foil hinges 364. In the compartment end, the rod 362 is attached by a hinge 365 placed at the attachment device 350, which is used to hold the membrane 348.

The pulling part 373 of the film is attached to a hinge 364 between the first 362 and the second rod 363 of the swivel arm, approximately halfway along its length. As the compartment 340 and the package 330 are moved towards each other as illustrated in FIG. 3b, the hinge between the first 362 and second 363 rod of swivel arm 361 will move upwards, thereby pulling the pulling part 373 of the film with it. At each end, the pulling part of the film converges into the covering parts, which is used to cover the inlet to the package and the outlet from the compartment respectively. FIG. 3c illustrates the situation where the inlet and the outlet are connected corresponding to the in-use position of the assembly.

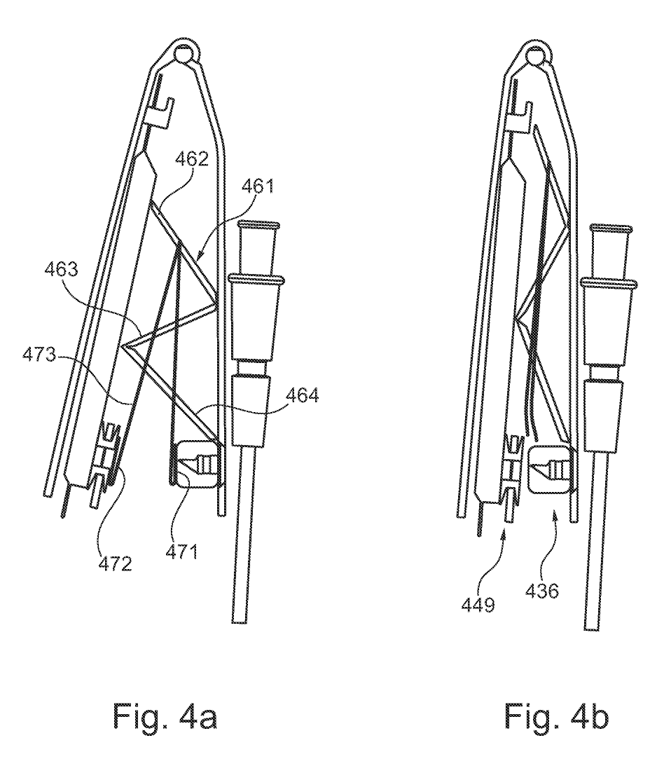

FIG. 4 illustrates an embodiment of the invention similar to the one in FIG. 3. The only differences are that the swivel arm 461 of this embodiment is left unattached at the first rod 462, and that the third rod 464 is longer. Furthermore, the pulling part of the film 473 is attached at the middle of the first rod 462 so that it will be farthest away from the outlet 449 and the inlet 436 respectively. It may also be attached to the top of the rod.

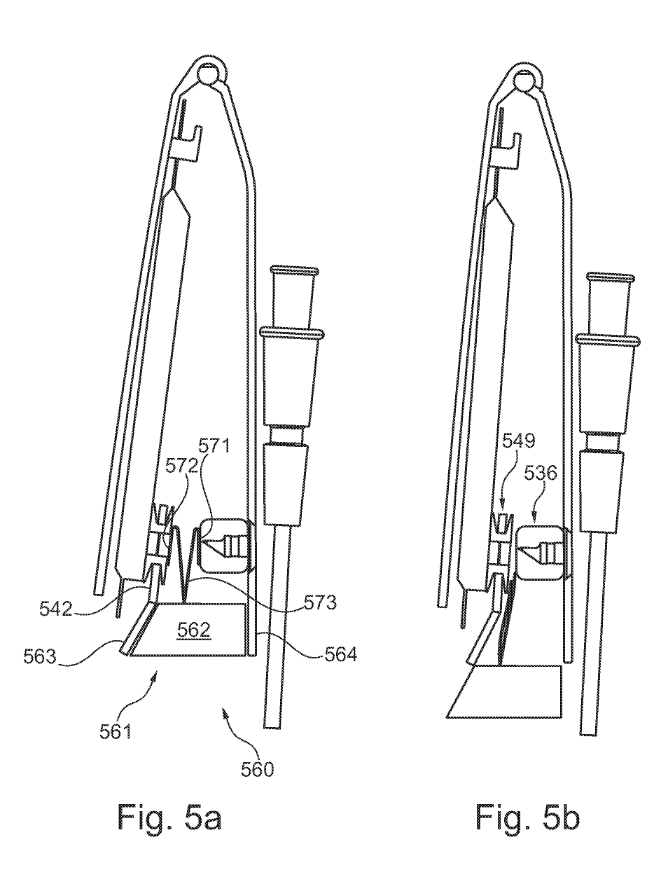

FIG. 5 illustrates an embodiment in which the film removal device 560 is in the form of a wedge mechanism 561. Again, FIG. 5a illustrates the embodiment in the storing-position, and FIG. 5b illustrates the embodiment in an intermediate position where the covering parts 571, 572 of the film are removed. Like with FIGS. 3 and 4, the parts that form no particular part of this embodiment have been left out. This mechanism comprises a wedge 562, which initially is maintained between two side elements, a first element 563 at the compartment side and a second element 564 at the package side. As illustrated, the first element 563 defines an angle with respect to the backside 542 of the compartment, although the first element 563 in this embodiment is integral with the backside 542 of the compartment. The angle may be approximately 150°, but any angle below 180° will work. In the illustrated embodiment, the cross-section of the wedge 562 is shaped as a trapezoidal element, but as mentioned earlier, it may also have other shapes such as cylindrical, spherical, and so on. The pulling part 573 of the film is attached to the wedge so that the pulling part is divided into two substantially equal parts.

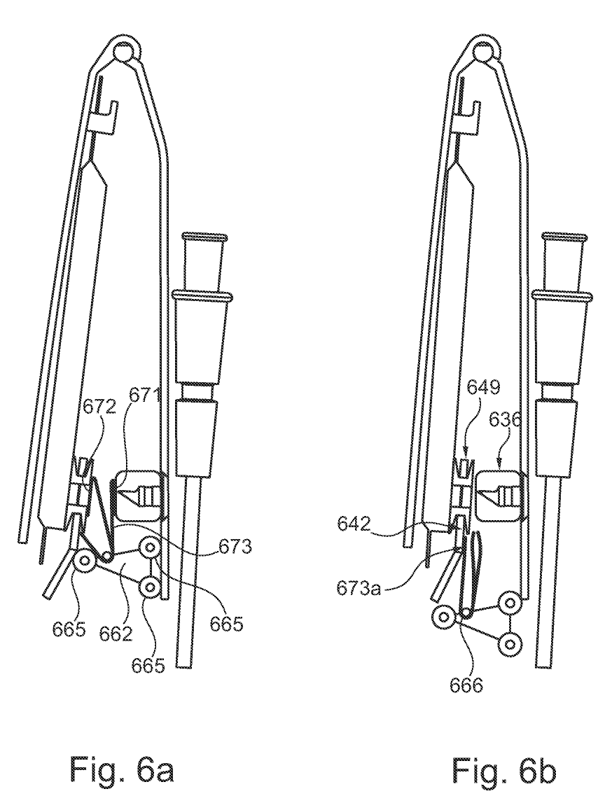

FIG. 6 illustrates an embodiment similar to the one in FIG. 5. The only differences are the configuration of the wedge 662 and the pulling part 673 of the film. In this embodiment, the wedge 662 is made as an element with a triangular cross-section, and with wheels 665 or rolls at the edges. Furthermore, the wedge 662 has a reel 666 around which the pulling part 673 of the film is rolled. The end 673a of the pulling part 673 of the film is attached to the backside 642 of the compartment just below the outlet 649 of the compartment.

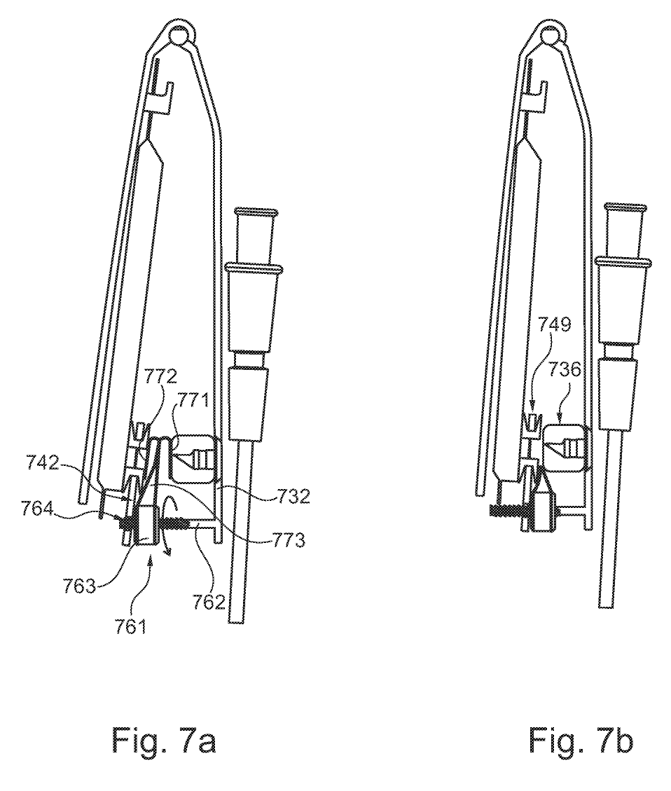

FIG. 7 illustrates an embodiment of the invention in which the film removal device 760 comprises a screw-and-spindle-construction 761. The spindle 762 extends from the rear 732 of the package and through a hole 764 in the rear 742 of the compartment. The hole 764 functions as a guide for the spindle to help maintain it in position during use. In other embodiments, the spindle may be rigid enough to be self-sustained. The rear of the compartment and the package are both extended downwards to make room for this device 760. In the illustrated embodiment, the screw 763 is configured to rotate in planes that are parallel to the backsides of the compartment and the package so that it rotates between them. However, the screw and spindle 761 may also be configured so that the screw 763 rotates in planes perpendicular to the back sides.

The end of the pulling part 773 of the film is wound up at the screw 763. As the compartment 740 is moved towards the package 730, the pulling part 773 of the film is wound up around the screw and the covering parts 771 and 772 are eventually removed from the inlet 736 and the outlet 749 respectively. This is illustrated in FIG. 7b.

1. An assembly for wetting a medical device comprising
a compartment for a fluid medium having an outlet
a package for containing the medical device comprising an inlet for the fluid medium,
where the package and the compartment are separate elements which are joined together,
the outlet and inlet are prior to use covered by covering parts of a barrier-film,
the assembly further comprises a film-removal device adapted to transfer a movement for bringing the outlet and inlet into contact with each other into a pulling force for removing the covering parts,
wherein the pulling force is exerted in a direction substantially transverse to the movement.

2. An assembly according to claim 1, wherein either the outlet from the compartment comprises a stud and the package inlet a membrane, or vice versa.

3. An assembly according to claim 1, wherein the film-removal device comprises a toothing mechanism.

4. An assembly according to claim 3, wherein the toothing mechanism comprises two sets of cooperating teeth facing each other, and where one set of teeth is attached to the compartment and another set of teeth is attached to the package.

5. An assembly according to claim 4, wherein two or more teeth have rounded tips facing each other.

6. An assembly according to claim 4, wherein two or more teeth comprise rolls at the tips facing each other.

7. An assembly according to claim 4, wherein the compartment has a top wall and a bottom wall and a length of the teeth adjacent the bottom wall is approximately twice as long as a length of the teeth adjacent the top wall.

8. An assembly according to claim 4, wherein the compartment has a top wall and a bottom wall and a length of the teeth adjacent the bottom wall is substantially the same as a length of the teeth adjacent the top wall.

9. An assembly according to claim 1, wherein the film-removal device comprises a swivel arm.

10. An assembly according to claim 9, wherein the swivel arm comprises two rods hinged together.

11. An assembly according to claim 9, wherein the swivel arm comprises more than two rods hinged together.

12. An assembly according to claim 9, wherein the swivel arm comprises a single hinged or bendable rod.

13. An assembly according to claim 10, wherein a first rod is attached near the compartment outlet and a second rod is attached near the package inlet.

14. An assembly according to claim 11, wherein a first rod is attached near the package inlet and a third rod is left unattached.

15. An assembly according to claim 1, wherein the film-removal device comprises a wedge mechanism.

16. An assembly according to claim 15, wherein the wedge mechanism comprises a wedge, a first side element on the compartment side and a second side element at the package side.

17. An assembly according to claim 16, wherein the first and second side elements are integral with a back side of the compartment and a back side of the package.

18. An assembly according to claim 16, wherein a cross-section of the wedge is shaped as a trapezoidal.

19. An assembly according to claim 16, wherein a cross-section of the wedge is shaped as a triangle with wheels or rolls at the edges.

20. An assembly according to claim 19 further comprising a reel at the wedge.

21. An assembly according to claim 1, wherein the film-removal device comprises a screw-and-spindle device.

22. An assembly according to claim 21, wherein a spindle of the screw-and-spindle device extends between the back side of the package and the back side of the compartment.

23. An assembly according to claim 21, wherein a screw of the screw-and-spindle device rotates in planes substantially parallel to the back side of the compartment and the package.

24. An assembly according to claim 23, wherein the screw rotates in planes substantially perpendicular to the back side of the compartment and the package.

25. An assembly according to claim 22, wherein the spindle defines a longitudinal direction, and where a screw thread at the spindle has an angle of approx 45° with respect to the longitudinal direction.

26. An assembly according to claim 22, wherein the spindle defines a longitudinal direction and where a screw thread at the spindle has an angle of approx 80° with respect to the longitudinal direction.

27. An assembly according to claim 23, wherein the screw is a ball-screw.

28. An assembly according to claim 1 further comprising a reel around which the pulling part of the barrier film is wound.

29. An assembly according to claim 1 further comprising a resilient element surrounding the stud at the package inlet.

30. An assembly according to claim 29, wherein the resilient element is a spring-element.

31. An assembly according to claim 29, wherein the resilient element is a sponge-like element.

32. An assembly according to claim 1, wherein the medical device is a urinary catheter with a hydrophilic coating.

33. An assembly according to claim 1, wherein the fluid medium comprises an antimicrobial agent including hydrogen peroxide.

34. A method for wetting a medical device stored in an assembly comprising a compartment for the fluid medium and a package for the medical device, where the compartment and the package are separate elements, which method comprises the steps of:
moving the compartment and the package with respect to each other from a position where the assembly is in a storing-position to a position where the assembly is in a use-position and
simultaneously removing covering parts of a barrier-film from an inlet to the package and an outlet from the compartment
where the removing of the covering parts is in a direction transverse to the moving of the compartment.

35. A method according to claim 34, wherein the removing of the covering parts is done by a toothing mechanism.

36. A method according to claim 34, wherein the removing of the covering parts is done by a swivel arm.

37. A method according to claim 34, wherein the removing of the covering parts is done by a wedge mechanism.

38. A method according to claim 34, wherein the removing of the covering parts is done by a screw-and-spindle device.

39. A method according to claim 34, wherein the medical device is a catheter and the fluid medium includes an anti-microbial agent.

40. A method for manufacturing an assembly that comprises:
assembling a package comprising an inlet and a part of a film-removal device and further including a medical device;
producing a compartment comprising an outlet and a part of a film-removal device and further including the fluid medium;
sterilizing the compartment and the package separately from each other.
joining the package and the compartment together;
covering the outlet and the inlet with covering parts of a barrier film; and
attaching a pulling part of the barrier film to the film-removal device.

41. A method according to claim 40, wherein the medical device has a hydrophilic coating.

42. A method according to claim 40, wherein sterilizing the package is done by radiation.

43. A method according to claim 40, wherein sterilizing the compartment is done by radiation.

44. A method according to claim 40, where the joining is done by attaching a hinge-part on the compartment to a hinge-part at the package.

45. A method according to claim 44, wherein one of the hinge parts is a hinge pin and the other a groove for receiving the hinge pin by clicking.
